(19) United States
(12) Patent Application Publication
(10) Pub. No.: US 2010/0299799 A1
(43) Pub. Date: Dec. 2, 2010

(54) CLOTHING ITEM WITH HETEROGENEOUS CONTENTION EFFECT FOR PRACTISING A SPORT

(75) Inventors: Nicolas Belluye, Lille (FR); Thibaud Thedon, Montpellier (FR); Kostia Puchaux, Lille (FR)

Correspondence Address: LADAS & PARRY LLP, 224 SOUTH MICHIGAN AVENUE, SUITE 1600, CHICAGO, IL, 60604, US

(73) Assignee: DECATHLON, Villeneuve D'Ascq (FR)

(21) Appl. No.: 12/599,531

(22) PCT Filed: Apr. 11, 2008
(86) PCT No.: PCT/FR2008/050641

§ 371 Date: Nov. 10, 2009

(30) Foreign Application Priority Data
May 11, 2007 (FR) 0755016

(51) Int. Cl. A41D 1/00; A61F 13/00; A41D 27/00

(52) U.S. Cl. 2/69; 602/62; 2/243.1

(57) ABSTRACT

A garment with localized compression effect for performing a sport, the garment being obtained by assembling first pieces of basic elasticity and second pieces that present a compression effect. The garment includes a plurality of types of second piece suitable for producing different compression effects, each type acting in a determined compression range; each second piece is arranged so as to produce a compression effect that is either longitudinal or transverse; and the second pieces of the various types are arranged in the assembly so as to surround certain transverse sections of at least one limb and/or the torso in full or in part and so as to exert a non-uniform compression effect with a pressure gradient that decreases towards the heart. In a variant, the second pieces are selected from three types, having compression ranges that correspond to compression effects that are respectively weak, medium, and strong.

The present invention lies in the technical field of garments presenting a localized compression effect, for practicing a sport, and more particularly garments obtained by assembling together first pieces of basic elasticity and second pieces penetrating a compression effect.

Compression is well known in the medical field, particularly for elastic socks or stockings for the purpose of exerting a continuously varying compression effect having a purpose that is therapeutic since it involves improving blood circulation, in particular for patients suffering from venous insufficiency. Those types of textile article are made of knitted elastic materials that are preferably knitted on circular knitting machines. Such textile articles are generally warm, unsightly, and unsuitable for practicing a sport since they put mechanical constraints on the movements of the user.

In the field of sport, it is known to exert a compression effect by using an elastic strap to surround either a joint in order to stabilize it, or muscle masses in order to support them. That compression technique is commonly referred to as “strapping”.

Document FR 2 879 900 in the name of the Applicant, describes tights presenting a localized effect for practicing a sport in which the legs perform repetitive motion, in particular in running or cycling races, the tights serving mainly to support muscle masses and to hold the knee joint, without requiring the user to generate forces additional to the forces inherently necessary for running or cycling races. The tights described in FR 2 879 900 do not serve to improve venous return or lymphatic drainage in any significant manner. Furthermore, that garment does not enable the racer's body to be maintained in an optimal position by limiting unwanted movements.

There is therefore a need for a garment that is adapted to practicing a given sports activity that involves particular movements of a repetitive nature, the garment enabling physiological, physical, and psychological fatigue to be reduced by simultaneously optimizing blood circulation, lymphatic drainage, proprioception, and supporting and holding muscle masses and joints, while correcting posture, and without giving rise to mechanical stresses that would be liable to constitute a source of additional effort over and above the movements required by said sports activity.

In order to satisfy the above-specified needs, the present invention provides a garment with localized compression effect for practicing a sport, the garment being obtained by assembling together first pieces of basic elasticity and second pieces presenting a compression effect, and the garment comprising, in characteristic manner, a plurality of types of second piece adapted to produce different compression effects, each type in a determined compression range; each of said second pieces is arranged in such a manner as to produce a compression effect that is either longitudinal or transverse; said second pieces of different types are arranged in the assembly in such a manner as to surround certain transverse sections of at least a limb and/or the torso either completely or in part and thereby exert a non-uniform compression effect with a pressure gradient that decreases going towards the heart.

The term second pieces having a “longitudinal” compression effect, is used to mean that said second pieces have the same transverse mean elasticity as the first pieces of basic elasticity, and that they have mean longitudinal elasticity that is less than half the mean longitudinal elasticity of the first pieces.

“Transverse” compression means that the second pieces have the same longitudinal mean elasticity as the first pieces of basic elasticity, the second pieces having mean transverse elasticity less than half the mean transverse elasticity of the first pieces.

The notion of mean elasticity takes account of the normal variations in the mechanical properties of any textile fabric, which variations are due to differences in particular in the origins of the raw materials, in weaving or knitting conditions, and in the settings of the looms or knitting machines. Variations about a mean value may be of the order of 5% to 10%, and sometimes even more.

A second piece of the present invention exerts a compression effect that is either longitudinal or transverse, but not both at once. An effect that is simultaneously transverse compression and longitudinal compression would act like a tourniquet and make it very difficult if not impossible to put the garment onto the limbs of the user.

Transverse compression is exerted substantially in the transverse direction of the limb in question or of the torso. Longitudinal compression is exerted substantially in the longitudinal direction of the limb in question or of the torso.

The term “limb” is used to mean a lower limb: thigh, leg, or foot, or an upper limb: upper arm, forearm, and hand.

The term “non-uniform” compression effect with a pressure gradient decreasing towards the heart means that all of the second pieces, each having a compression effect over a given compression range, are arranged in the assembly in such a manner that the sequence of compression values applied by the succession of said second pieces decreases going from the distal end of the limb or the trunk towards its proximal end. This arrangement of the second pieces serves advantageously to increase venous return and lymphatic drainage.

Depending on the sports activity being practiced, the garment of the present invention advantageously presents a transverse compression effect to support a joint, to hold muscle masses, and to improve blood circulation, lymphatic drainage, and proprioception, and a longitudinal compression effect to guide movements, and to reduce unwanted movements so as to keep the limb or the torso on which it is applied in an optimum position.

The user is not impeded in practicing a given physical activity by the first pieces of basic elasticity, nor by the second pieces presenting basic elasticity in the longitudinal or transverse direction depending on the movements being performed.

In addition, since the second pieces exert compression effects over different compression ranges, it is possible to reinforce the supporting and/or holding of muscle masses and of joints and the improvement of blood circulation, lymphatic drainage, and proprioception depending on the zones of the body under consideration and on the muscles used to optimize these various functions for practicing a given sports activity.

In a variant, the second pieces are selected from three types having compression ranges corresponding to compression effects that are respectively weak, medium, and strong.

It is thus possible to modulate the transverse or longitudinal compression effects in zones of the body that are particularly stressed during a given sports activity.

In a variant, the first type of weak compression effect corresponds to a pressure range of [10, 15] millimeters of mercury (mmHg), the second type of medium compression effect corresponds to a pressure range of [15, 20] mmHg, and the third type of strong compression effect corresponds to a pressure range of [20, 25] mmHg.

The compression ranges are determined by applying principles of muscular compression. In a variant, the garment of the present invention is in the form of a sleeve on its own, comprising a forearm portion and an upper arm portion.

In a subvariant, the forearm portion comprises a second piece of transverse compression effect covering at least the finger flexor muscles. Preferably, said second piece of transverse compression effect exerts pressure on the finger flexor muscles lying in a medium compression effect range, preferably in the range [15, 20] mmHg.

In a sub-variant, the upper arm portion includes a second piece of transverse compression effect covering at least the biceps and triceps brachii muscles. Preferably, said second piece of transverse compression effect exerts pressure on the biceps and triceps brachii muscles lying in a weak compression effect range, and preferably in the range [10, 15] mmHg.

The pressure exerted transversely on the finger flexor muscles and the biceps and triceps brachii muscles serves to increased venous return and thus to avoid tingling feelings and the heavy arm sensation. Tingling feelings, also known as “pins and needles”, occur when normal blood pressure is reestablished following a reduction in the rate of blood irrigation when one or more muscles have been compressed, thereby giving rise to temporary extra pressure at a constriction. Thus, the transverse compression on the above-mentioned muscles is particularly advantageous when practicing activities in mountains and in particular climbing, where the above-mentioned muscles are contracted and sometimes compressed, the upper arm often being in extension and supporting the weight of the body.

Furthermore, depending on altitude, there can be observed a decrease in the partial pressure of oxygen between arteries and veins, and as atmospheric pressure decrees, the pressure in the body relaxes and decreases, with muscle tissue and organs being less irrigated, so the subject has less energy. Thus, the transverse compression exerted by said second pieces can also make it possible to combat this phenomenon while being active in mountains at medium altitude, in particular below 4000 meters.

A said garment also includes first pieces of basic elasticity that do not exert any particular compression effect since the effect is less than 7 mmHg, thereby leaving the user completely free in the movements that can be made.

In a subvariant, the garment includes anchoring elements at its ends and at the elbow joint.

Preferably, these anchor elements are located close to the wrist, to the top of the arm, and to the elbow, and they comprise elastic bands enabling the sleeve on its own to be kept in place on the moving user. In a variant, the garment of the present invention is in the form of a long-sleeved top having a forearm portion, an upper arm portion, a chest portion, a shoulder portion, a back portion, and an abdomen portion.

In a subvariant, the shoulder portion includes a first piece of basic elasticity covering the deltoid muscle and the shoulder blade.

The garment has two shoulder portions that are symmetrical about the longitudinal axis of the torso. Said first piece of basic elasticity does not deliver any additional mechanical stress, whether in the transverse or the longitudinal direction of the torso, and thus gives rise to no requirement for additional effort by the user. The user thus benefits from being able to move the shoulder joint with full amplitude. This disposition is particularly advantageous for swimming and surfing.

In a subvariant, the abdomen portion comprises a first piece of basic elasticity covering the abdominal muscles.

This arrangement serves to avoid impeding the user's breathing.

In a subvariant, the chest portion includes a second piece of transverse compression effect covering the greater pectoral muscle.

The pectoral muscles are thus supported and held, in particular while swimming or surfing, thereby improving the propulsion action of the body. Said second piece also makes it possible to improve venous return to the heart and in particular to improve oxygenation of muscle tissues.

In a subvariant, the back portion includes a second piece of transverse compression effect covering the broad muscle of the back.

The garment has two back portions that are symmetrical about the longitudinal axis of the torso. Said second piece improves venous return and supports the broad muscle of the back in its propulsion action, in particular when swimming or surfing. Said second piece also corrects posture by keeping the back in extension and by limiting unwanted movements.

Preferably, said second piece of transverse compression effect is extended by a second piece of longitudinal compression effect over the top of the back which piece is shared between opposite sides of the nape of the neck, in particular to cover the trapezius and rhomboid muscles.

Preferably, said second piece covers the projecting portions of the collar bone. The extension of the second piece over the nape of the neck enables the neck to be kept extended and encourages propulsion action during swimming.

In a subvariant, the abdominal portion includes a second piece of transverse compression effect covering the external oblique muscle and the bottom of the rectus muscle.

Said second piece has in particular the function of supporting the stabilizing action of the trunk, of attenuating inclination of the upper body in the sagittal plane, and attenuating rotations, so as to maintain an optimum position for swimming.

In a subvariant, the second pieces covering the greater pectoral muscle, the broad muscle of the back, the external oblique muscle, the rectus muscle, the trapezius muscles, and the rhomboid muscles, exert pressure lying in a weak compression effect range, preferably in the range [10, 15] mmHg.

In a subvariant, the forearm portion includes a second piece of transverse compression effect covering the finger flexor muscles and exerting pressure lying in a strong compression effect range, preferably in the range [20, 25] mmHg.

In a subvariant, the upper arm portion includes a second piece of transverse compression effect covering the biceps and triceps brachii muscles and exerting pressure lying in a weak compression effect range, and preferably in the range [10, 15] mmHg.

The second pieces placed on the forearm portion and the upper arm portion have the function of improving venous return, reducing tingling feelings and reducing “heavy arm” sensations.

In a subvariant, the second piece of transverse compression effect on the back portion includes a mock seam so as to press said garment on the user's back, preferably along the spinal column.

The mock seam also serves to conserve a high level of compression on the nape of the neck in spite of the size of the second piece.

In a subvariant, said garment includes third pieces for covering the armpits and in the form of nets (fabric having a very open mesh) to encourage evacuating heat and perspiration.

The nets are preferably elastic and do not impede the movements of the user.

In a variant, the garment of the present invention is in the form of tights comprising an anterior thigh portion, a posterior thigh portion, a knee portion, and a leg portion.

In a subvariant, the anterior thigh portion includes a second piece of transverse compression effect covering the rectus muscle, the lateral vastus muscle, and the adductor muscles or the medial vastus muscle.

For swimming, the second piece preferably covers the adductor muscles and the medial vastus muscle so as to increase blood flow to the heart and reduce tingling feelings and “heavy leg” sensations.

For surfing, said second piece compresses the medial vastus muscle but not the adductor muscles so as to increase the blood flow to the heart and improve warming up and recovery.

In a subvariant, the posterior portion of the thigh includes a second piece of transverse compression effect covering the femoral biceps muscles and the adductor muscles or the semi-membraneous muscles.

For surfing, the semi-membraneous muscles are compressed but not the adductor muscles so as to improve warming up and recovery.

For swimming, the adductor muscles are compressed but not the semi-membraneous muscles, so as to improve venous return and decrease cramps and tingling feelings.

In a subvariant, the knee portion includes a second piece of transverse compression effect covering with its anterior face the head of the fibula and with its posterior face the inside of the knee joint so as to extend over the top portion of the gastrocnemius, passing under said knee joint.

This second piece is thus provided in particular for swimming. It serves to support and guide the flexing and extending work of the knee while propelling the swimmer.

Preferably, said second pieces of longitudinal or transverse compression effect exert pressure lying in a weak pressure effect range, and preferably in the range [10, 15] mmHg.

In a subvariant, the leg portion includes a second piece of transverse compression effect covering the gastrocnemius muscles and the toe extensor muscles.

When swimming, this provision enables blood flow to the heart to be increased and reduces tingling feelings and cramps. For surfing, a greater improvement is obtained in recovery and warming up.

Preferably, said second piece of transverse compression effect exerts pressure lying in a strong compression effect range, and preferably in the range [20, 25] mmHg.

In a subvariant, said garment has third pieces, in particular open mesh pieces, e.g. in the form of a net, to cover the popliteal cavity of the knee and to encourage evacuating heat and perspiration.

In a variant, the garment of the present invention is in the form of a long-sleeved costume, in particular for swimming, comprising a forearm portion, an upper arm portion, a chest portion, a shoulder portion, a back portion, an abdomen portion, a buttocks portion, a thigh anterior portion, a thigh posterior portion, a knee portion, and a leg portion according to any above-described embodiment.

In a subvariant, said back portion includes a second piece of longitudinal compression effect covering the erector muscles of the spine and extending towards the bottom of the back over the bottom portion of the luteal muscle and the tensor of the fascia lata, and towards the top of the back over the spinal column so as to be shared on either side of the nape of the neck and cover in particular the trapezius and rhomboid muscles. Said second piece exerts pressure lying in a weak compression effect range, and preferably in the range [10, 15] mmHg. In a variant, the abdomen portion includes a second piece of longitudinal compression effect covering, on passing below the navel on either side of the waist, the internal oblique muscle and the rectus muscle. Said second piece exerts pressure lying in a weak compression effect range, and preferably in the range [10, 15] mmHg. The above-mentioned muscles are supported by said second piece in their action of stabilizing the upper body. Said second piece serves to attenuate the inclination of the upper body in the sagittal plane and to maintain an optimum position, in particular for swimming. The description of the arrangement of the first, second, and third pieces of the forearm, upper arm, chest, shoulder, buttocks, anterior and posterior thigh, knee, and leg portions of the costume is not repeated since it corresponds to the arrangements described in particular for the long-sleeved top and for the tights for swimming.

In a variant, the garment includes a first piece of basic elasticity covering the elbow joint, and preshaped with an angle of 45° corresponding to the natural bending angle of the elbow.

The present invention can better understood on reading the following description of embodiments given in non-limiting manner and shown in the following figures accompanying the present specification:

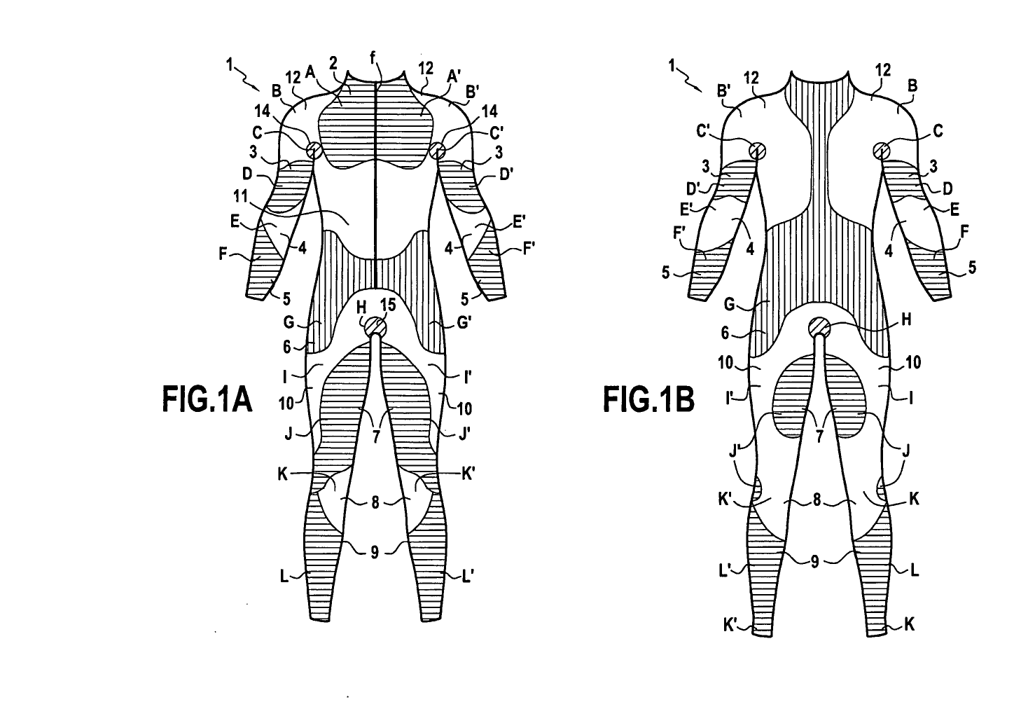

FIGS. 1A and 1B are respectively a front view and a back view of a costume for swimming;

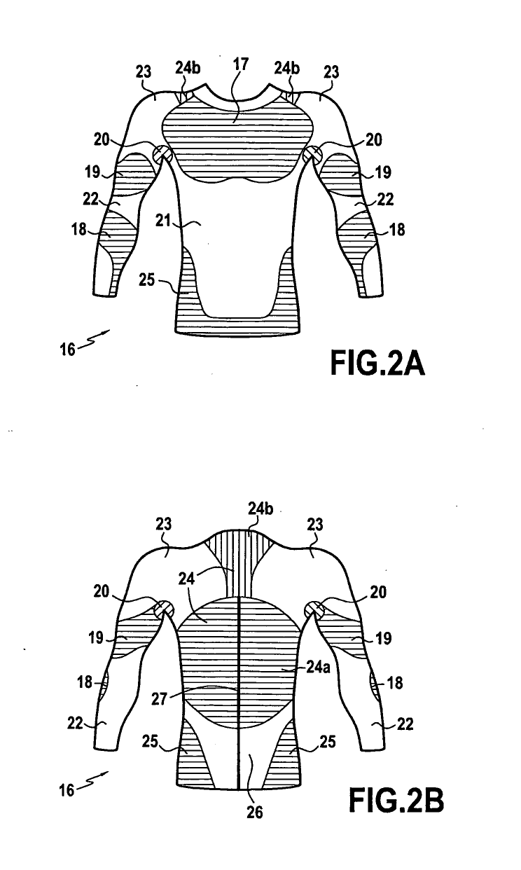

FIGS. 2A and 2B are respectively a front view and a back view of a first example of a long-sleeved top for swimming or surfing;

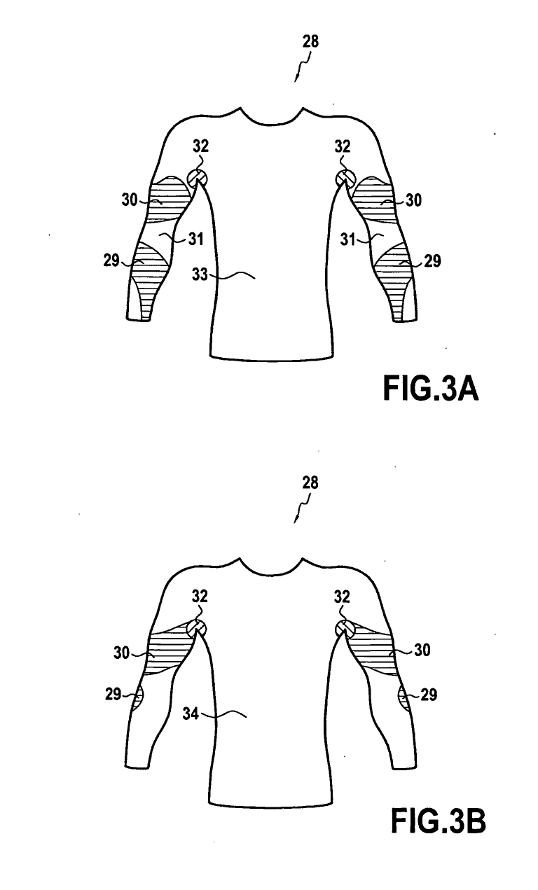

FIGS. 3A and 3B are respectively a front view and a back view of a second example of a long-sleeved top for surfing or climbing;

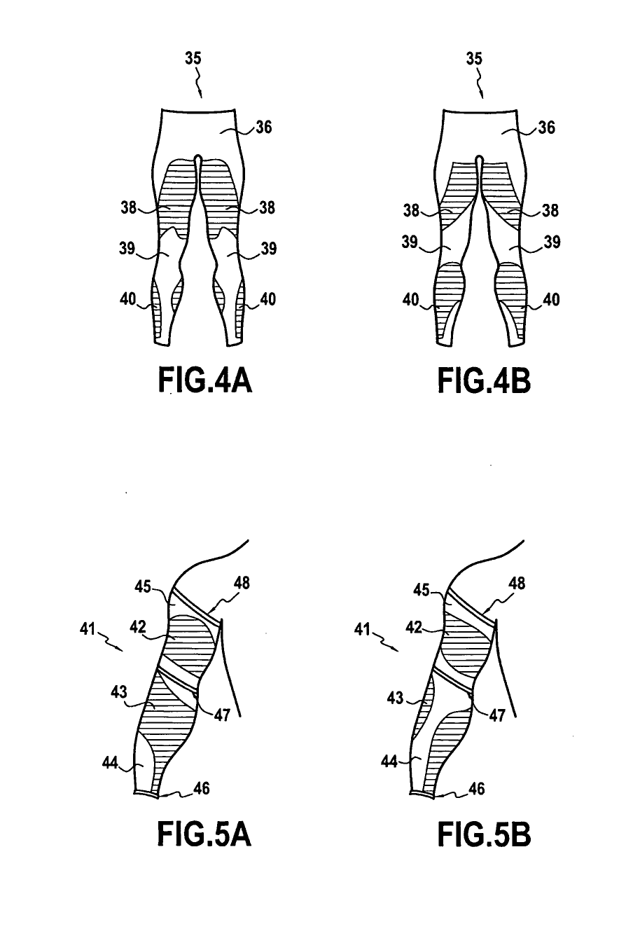

FIGS. 4A and 4B are respectively a front view and a back view of tights for surfing or swimming;

FIGS. 5A and 5B are respectively a front view and a back view of a sleeve on its own for climbing and/or mountaineering; and

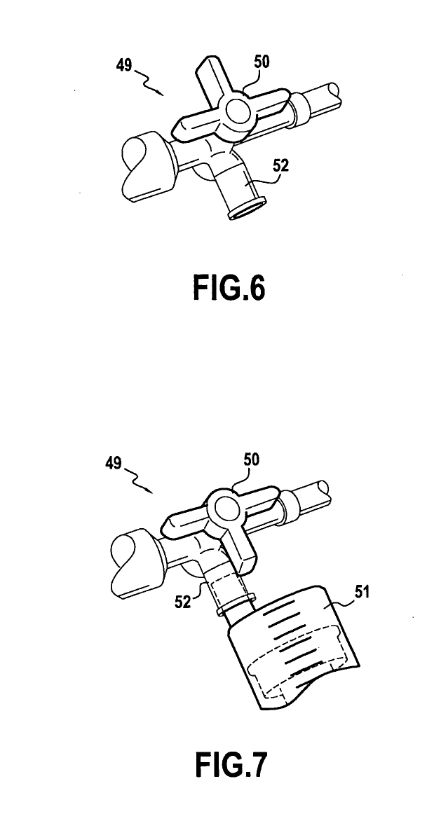

FIGS. 6 and 7 are diagrammatic views of the Kikuhime type pressure-measurement apparatus.

The garments of the present invention, in particular a costume, a long-sleeved top, or a sleeve on its own are constituted by assembling first, second, and third pieces using a technique that limits or eliminates risks of non-uniform elasticity between the assembled-together pieces, as explained below. The edges of the assembled-together pieces are juxtaposed without any superposition. The pieces referred to below as “first” pieces are cut from an elastic material, presenting normal elasticity. For example, it may be a ladderproof knit, using the so-called charmeuse locknit, weighing about 170 grams per square meter (g/m²), and constituted by an 80/20 polyamide/spandex yarns. The pieces referred to be low as “second” pieces are cut from an elastic textile material—possibly including an elastomer, e.g. neoprene—having mean elasticity in the transverse or longitudinal direction that is well below that of the first pieces, by a ratio of at least 1 to 2.

The second pieces are preferably cut out from a single elastic material. By reducing the quantity of material forming the second pieces in the garment to a greater or lesser extent depending on the size to which the garment is to correspond, once the garment is being worn the second pieces exert a compression effect that is weak, medium, or strong.

To measure the mean elasticity, elongations are calculated under a force of 15 newtons (N). In the above-mentioned examples, the mean elasticities of the first and second pieces in one of the longitudinal or transverse directions, depending on the type of compression that is desired, are practically identical, with a difference of less than 5%, whereas the mean elasticities between the first and second pieces in the other one of the longitudinal or transverse directions differ by a ratio of not less than 2.

The pieces referred to below as “third” pieces are pieces of mean elasticity greater than that of the first piece. They comprise in particular a net having a relatively open mesh, of the order of a few millimeters, also serving to provide an aeration effect for the zones they cover.

Assembly is performed by any conventional technique, in particular by stitching, adhesive, or heat-sealing. With a cut-and-sewn assembly, the stitching is preferably of the flatlock or overcasting type, using crimped yarn, e.g. textured by a weft knitting machine. Under such circumstances, it is desirable to adjust the tension of the threads in the sewing machines to a value that is less than that commonly used so as to limit the negative effect that might be presented by the stitching on the uniformity of the mean elasticities of the assembled-together pieces.

Furthermore, the number of panels used in making the assembly may vary, in particular depending on whether the garment is for a man or a woman, and what matters is not the number of panels, but rather how the first pieces, the various types of second piece, and the third pieces are distributed over the portions of the body concerned by said garment.

In the figures, the panels forming the first pieces having basic elasticity are shown without shading, the panels forming the second pieces with a transverse compression effect are shown with transverse shading, the panels forming the second pieces with a longitudinal compression effect are shown with longitudinal shading, and the panels forming the third pieces are shown with oblique shading.

In the description below, the term “panel” is used in the detailed description of the embodiments shown in the figures, and the terms “first”, “second”, and “third” piece are used concerning their specific locations over zones of the body.

The costume 1 shown in FIGS. 1A and 1B is particularly adapted for swimming. It is made by assembling together twenty-two or twenty-three panels consisting of eight panels [B, B′, E, E′, I, I′, K, K′] having the structure of first pieces having basic elasticity, ten panels [A, A′, D, D′, F, F′, J, J′, L, L′] having the structure of second pieces having a transverse compression effect, a panel [G] having the structure of second pieces having a longitudinal compression effect, and two [or three] panels C, C′, [and possibly H] having the structure of third pieces, and made of an open-mesh net. The panels [A, A′, D, D′, F, F′, J, J′, L, L′] have mean transverse elasticity lower than that of the panels [B, B′, E, E′, I, I′, K, K′] and the panel [G] has longitudinal mean elasticity lower than that of the panels [B, B′, E, E′, I, I′, K, K′].

In the top portion of the costume 1, in FIG. 1A, a second piece 2 of transverse compression effect covers the greater pectoral muscle. The second piece 2 is made up of two panels A and A′ that are symmetrical about a zip fastener f, enabling the costume to be put on and going down as far as the navel. The greater pectoral muscle serves to cross, raise, and lower the arms in front of the chest and this may optionally be done in rotation. The second piece 2 has the function of holding and supporting the greater pectoral muscle in its action of propelling the body while swimming, and also of increasing venous return.

The second piece 3 made of panels D and D′ covers the biceps and triceps brachii muscles (long head, vastus laterales, vastus medialis).

The second piece 5, made up of panels F and F′, covers the finger flexor muscles. The biceps and triceps brachii muscles act in coordination to raise and lower the forearm. The second piece 3 extends over the posterior portion above the elbow as far as the armpit, and it completely surrounds the anterior portion of the arm while avoiding the fold of the elbow. The extensions of the triceps and biceps trachii muscles on the shoulder blade are not covered by the second piece 3 in order to avoid impeding movements of the shoulder. The triceps brachii serves to extent the elbow, it controls raising and lowering the forearm together with the biceps brachii.

The second pieces 3 and 5 exert a transverse compression effect so as to encourage venous return, reduce tingling feelings and reduce the “heavy arm” sensation.

In FIG. 1B, the second piece 6 having a longitudinal compression effect, formed by the panel G, covers the bottom portion of the broad muscle of the back down to the top of the gluteal muscles. The second piece 6 does not cover the left and right collar bones. The broad muscle of the back is a very large flat muscle covering the bottom half of the back. It moves the arms and it is particularly used while swimming. The second piece 6 supports the action of the broad muscle of the back propelling the body, thereby contributing to holding the back extended and encouraging venous return.

The second piece 6 extends from the back towards the abdomen and covers the external oblique muscles of the abdomen, the bottom of the rectus muscle, the erector muscles of the spine, and extends towards the bottom of the gluteal muscle and the tensor muscle of the fascia lata. This second piece 6 is formed in this example of a single panel G going all the way around the swimmer's pelvis. The rectus muscle serves to bend the upper body forwards and also serves to tension the abdominal wall and compress the internal organs. The external oblique muscle of the abdomen is large and near the surface, extending laterally over the front of the trunk. It comprises two portions: a top, chest portion and a bottom, lateral portion, which portions meet laterally on the pelvis. The external oblique muscle serves to bend the trunk forwards and to turn the upper body to one side or the other. The gluteal muscle extends downwards and outwards covering the lateral and posterior projections of the pelvis. The gluteal muscle is an extensor of the thigh at the hip joint and it serves to pull it rearwards while the legs are moving up and down while swimming. The tensor of the fascia lata is a small elongate muscle that extends from the iliac bone to the greater trochanter. It enables internal rotation of the femur, and it also serves as an adductor flexor for the thigh towards the pelvis. By its longitudinal compression effect, the second piece 6 supports the action of the muscles it covers in their function of stabilizing the trunk, and it attenuates the forward inclination of the upper body so as to maintain an optimal position while swimming.

The first pieces 11 and 12 of basic elasticity respectively cover the abdominal muscles and the deltoid muscle and the scapula (shoulder blade). The first piece 12 does not oppose the movements of the swimmer's arm and permits large-amplitude movements of the shoulder joint. The first piece 11 leaves abdominal breathing movements free. The first pieces 11 and 12 in this example 1 are formed by the panels B and B′.

The second piece 7 having transverse compression effect covers the anterior face of the thigh, covering the rectus muscle, the lateral vastus muscle, the medial vastus muscle, and the adductors. The second piece 7 extends inwards and upwards to the top portion of the thigh, outwards and downwards to terminate beside the knee joint, and inwards and downwards to cover the knee joint (hamstring). The above-mentioned muscles on the posterior face of the thigh act together to extend the knee. On the posterior face of the thigh, the second piece 7 covers the femoral biceps muscle and the adductors. The second piece 7 is formed two panels J and J′, and has the function of improving venous return, decreasing tingling feelings and “heavy leg” sensations, and also decreasing cramp.

At the knee, the second piece 7 passes on the outside and terminates at the head of the fibula. The second piece 7 also passes over the inside of the knee joint (hamstring) and extends over the posterior portion, passing under the cavity of the back of the knee joint and covers the top portions of the gastrocnemius muscles: This disposition of the second piece 7 serves to support and guide the work of the knee in flexing and extending while propelling the swimmer.

The second piece 9 with a transverse compression effect covers the gastrocnemius muscles and the toe extensor muscles so as to increase venous return, decrease tingling feelings and cramp. The second piece 9 is formed of two panels L and L′.

The first piece 4 of basic elasticity, formed by the two panels E and E′ covers the elbow joint and the popliteal cavity of the elbow. The two panels E and E′ are made of material having basic elasticity preformed at an angle of 45° corresponding to the natural bending angle of the elbow. No mechanical stress is thus exerted on the elbow joint and movements of the forearm are not impeded.

Another first piece 10 of basic elasticity, formed of panels I and I′, covers the posterior and anterior portions of the thigh in part, and in particular the iliotibial tract and extends towards the bottom edge of the second piece 6.

A first piece 8 of basic elasticity, formed of panels K and K′, covers the anterior face of the leg, the patella (or knee cap), and its top and bottom peripheries. The second piece 9 extends towards the foot, in particular covering the lateral tibial muscle and the tibia.

The third pieces 14 in the form of an open-mesh net, formed by the panels C and C′ cover the armpits to evacuate heat and perspiration. A third piece 15 formed by the panel H optionally covers the perineum.

The first pieces 4, 8, 10, 11, and 12 of basic elasticity in this example 1 have a compression effect of less than 7 mmHg.

The second piece 5 exerts a strong compression effect, preferably lying in the range [20, 25] mmHg. The second pieces 3 and 2 exert a weak compression effect, preferably lying in the range [10, 15] mmHg. Thus, the second pieces 2, 3, and 5 exert a discontinuous compression effect with a pressure gradient that decreases from the distal end of the limb, here the forearm, towards the heart.

Likewise, the second piece 9 exerts a strong compression effect, preferably lying in the range [20, 25] mmHg. The second pieces 6 and 7 exert a weak compression effect, preferably lying in the range [10, 15] mmHg.

An increase of venous return is thus well ensured, thereby enabling tissues to be better oxygenated and reduces tingling feelings and “heavy limb” sensations.

By compressing the body locally to a greater or lesser extent in the longitudinal or the transverse direction, the costume 1 improves blood circulation, lymphatic drainage, and proprioception. It holds muscle masses and stabilizes the joints to protect against injury, guides movement, reduces unwanted movements, and maintains a hydrodynamic position in water.

In the description of the first, second, and third pieces below, mention is no longer made of the panels proper, which relate to how the garment is assembled while it is being made, but solely to the first, second, and third pieces that are characteristic of the present invention.

In the second example as shown in FIGS. 2A and 2B, the long-sleeved top 16 is intended in particular for swimming and surfing. This top 16 presents differences relative to the arrangement of the first, second, and third pieces located on the top of the body of the costume 1 for swimming.

The description below seeks to emphasize these differences.

The second pieces 17, 18, and 19 are equivalent respectively to the second pieces 2, 3, and 5 of the costume 1. The first pieces 21, 22, and 23 are equivalent respectively to the first pieces 11, 4, and 12 of the costume 1. Similarly, the third piece 20 is equivalent to the third piece 14 of the costume 1.

The second piece 24 comprises two portions, a first portion 24a having a transverse compression effect and covering the broad muscle of the back and that is extended over the top of the back by a second portion 24b having a longitudinal compression effect that is shared on either side of the nape of the neck to cover in particular the trapezius and rhomboid muscles. The second piece 24 has the functions of improving venous return, supporting the broad muscle in its action in propelling the body, in particular while swimming, and facilitating keeping the neck extended. The second piece 24 is subdivided into two substantially symmetrical portions by a mock seam 27 starting from the top edge of the second piece 24a and going down to the gluteal muscles along the spinal column. The mock seam 27 serves to hold the second piece 24b and the first piece 26 pressed against the back.

The second piece 25 presents transverse compression, covering, on the abdomen, the external oblique muscle and the bottom of the rectus muscle. Its function is to increase venous return, to support said muscles in their action of stabilizing the trunk, and to attenuate the inclination of the upper body in the sagittal plane so as to maintain an optimum position, in particular while swimming.

The second piece 18 exerts a strong compression effect, preferably lying in the range [20, 25] mmHg. The second pieces 17, 19, and 24 exert a weak compression effect, preferably lying in the range [10, 15] mmHg. The pressure exerted is thus degressive from a distal end, i.e. the wrist, towards the proximal end, i.e. the heart.

The top 16 holds the upper body, the shoulders, and the nape of the neck in an ideal position for facilitating breathing and encouraging the propulsive action of the movement of the arms. Like the costume 1, it also serves to improve blood circulation, lymphatic drainage, and proprioception. By holding the muscle masses, the joints are stabilized and the risk of injury is reduced.

The long-sleeved top 28 shown in FIGS. 3A and 3B is particularly adapted to surfing or climbing.

The second pieces 29 and 30 are equivalent respectively to the second pieces 5 and 3 of the costume 1, and by applying a strong compression effect, preferably in the range [20, 25] mmHg, they also serve to increase venous return so as to improve warming up and recuperating after physical activity.

The third piece 32 is equivalent to the third piece 14 of the costume 1. The first piece 31 is equivalent to the first piece 4 of the costume 1. The top 28 in this example has two first pieces 33 and 34 of basic elasticity respectively covering the anterior and posterior faces of the torso. The first pieces 31, 33, and 34 exert a very weak compression effect, preferably less than 5 mmHg, so as to avoid impeding the amplitude of a surfer's movements.

The tights 38 shown in FIGS. 4A and 4B are particularly intended for surfing or swimming, and they may be combined with the above-described tops 16 and 28.

The second piece 40 is equivalent to the second piece 9 of the costume 1. The first piece 39 of basic elasticity covers in particular the head of the fibula and the popliteal cavity of the knee so as to allow great amplitude of joint movement. The first piece 36 of basic elasticity covers the gluteal muscles and the iliotibial tract. The second piece 38 presenting transverse compression extends down to the gluteal fold covering, on the anterior face of the thigh: the lateral vastus muscle, the rectus muscle of the thigh, and the medial vastus muscle; and on its posterior face: the femoral biceps muscle and the semi-membranous muscle.

The second piece 40 exerts a strong transverse compression effect, preferably lying in the range [20, 25] mmHg. The second piece 38 exerts a medium transverse compression effect, preferably lying in the range [10, 15] mmHg.

The first pieces 36 and 39 of basic elasticity exert a very weak compression effect, preferably less than 5 mmHg. The tights 35 are adapted to improve recovery and warming up when performing physical activities, and they also increase venous return.

The sleeve 41 shown in FIGS. 5A and 5B is particularly intended for climbing and/or mountaineering activities.

The second pieces 42 and 43 having a transverse compression effect are equivalent respectively to the second pieces 3 and 5 of the costume 1, except that the second piece 43 exerts a medium compression effect, preferably lying in the range [15, 20] mmHg. The first pieces 44 and 45 of basic elasticity are preformed at an angle 45° corresponding to the natural bending angle of the elbow. Furthermore, the first pieces 44 and 45 exert a very weak compression effect, less than 7 mmHg. The sleeve 41, also referred to as an “armband”, has three anchoring elements in the form of elastic tapes at its ends 46 and 48 and at the elbow joint 47. These elements 46, 47, and 48 enable the armband to be held on the arm. The sleeve serves to increased venous return, to reduce tingling feelings, and to reduce the “heavy arm” sensation.

The method of operation described below serves to measure the pressure in mmHg exerted by a garment on a given zone of the body with the help of a Kikuhime type pressure sensor. This method is mentioned in non-limiting manner and is used for measuring the pressures exerted by the first, second, and third pieces forming garments of the present invention.

The first step comprises calibrating the Kikuhime pressure sensor apparatus 49, as shown in FIGS. 6 and 7, and is necessary prior to performing any pressure measurements, and in particular it comprises the following steps:

1) connecting the airbag to the unit;

2) opening the cock 50;

3) connecting the syringe 51 to the port 52 of the cock;

4) pushing or pulling on the syringe 51 until the value displayed by the sensor indicates “0”;

5) closing the port of the port 52 of the cock in which the syringe 51 is connected;

6) removing the syringe 51; and

7) the sensor is calibrated.

The second step is measuring the pressure exerted by a garment on a given zone of the body, and it comprises the following steps in particular:

1) making a mark on the skin where the measurement is to be taken;

2) the subject putting on the garment for testing, which garment is then in an initial position;

3) lifting said garment on the limb or the torso so as to be able to see said mark;

4) placing the sensor on said mark;

5) replacing the garment in its initial position;

6) verifying that the sensor has not slipped or turned over;

7) placing the subject in a reference position, without moving, and more particularly standing for measurements performed on garments of the present invention;

8) measuring the pressure displayed on the sensor (NB.: the tolerance of the measurement apparatus 49 is ±1 mmHg, according to the manufacturer); and

9) performing this manipulation twice over to ensure that the measurement is reproducible.

1. A garment with localized compression effect for performing a sport, the garment being obtained by assembling first pieces of basic elasticity and second pieces that present a compression effect, the garment comprising:
a plurality of types of second piece suitable for producing different compression effects, each type acting in a determined compression range;
wherein each second piece is arranged so as to produce a compression effect that is either longitudinal or transverse; and
wherein the second pieces of the various types are arranged in the assembly so as to surround certain transverse sections of at least one limb and/or the torso in full or in part and so as to exert a non-uniform compression effect with a pressure gradient that decreases towards the heart.

2. A garment according to claim 1, further including second pieces selected from three types, having compression ranges that correspond respectively to weak, medium, and strong compression.

3. A garment according to claim 2, (wherein) the first type of weak compression effect corresponds to a pressure range of 10-15 mmHg, the second type of medium compression effect corresponds to a pressure range of 15-20 mmHg, and the third type of strong compression effect corresponds to a pressure range of 20-25 mmHg.

4. A garment according to claim 1, wherein the garment is in the form of a sleeve on its own, comprising a forearm portion and an upper arm portion.

5. A garment according to claim 4, wherein forearm portion comprises a second piece of transverse compression effect covering at least the finger flexor muscles.

6. A garment according to claim 5, wherein said second piece of transverse compression effect exerts pressure on the finger flexor muscles lying in a medium compression effect range.

7. A garment according to claim 4, wherein the upper arm portion includes a second piece of transverse compression effect covering at least the biceps and triceps brachii muscles.

8. A garment according to claim 7, wherein said second piece of transverse compression effect exerts pressure on the biceps and triceps brachii muscles lying in a weak compression effect range.

9. A garment according to claim 4, further comprising it includes anchoring elements at ends of the garment and at an elbow joint.

10. A garment according to claim 4, further comprising it is in the form of a long-sleeved top having a forearm portion, an upper arm portion, a chest portion, a shoulder portion, a back portion, and an abdomen portion.

11. A garment according to claim 10, wherein the shoulder portion includes a first piece of basic elasticity covering the deltoid muscle and the shoulder blade.

12. A garment according to claim 10, wherein the abdomen portion comprises a first piece of basic elasticity covering the abdominal muscles.

13. A garment according to claim 10, wherein the chest portion includes a second piece of transverse compression effect covering the greater pectoral muscle.

14. A garment according to claim 10 wherein the back portion includes a second piece of transverse compression effect covering the broad muscle of the back.

15. A garment according to claim 14, wherein said second piece of transverse compression effect is extended by a second piece of longitudinal compression effect over the top of the back, which piece is shared between opposite sides of the nape of the neck, in particular to cover the trapezius and rhomboid muscles.

16. A garment according to claim 10, wherein the abdominal portion includes a second piece of transverse compression effect covering the external oblique muscle and the bottom of the rectus muscle.

17. A garment according to claim 10, wherein the second pieces covering the greater pectoral muscle, the broad muscle of the back, the external oblique muscle, the rectus muscle, the trapezius muscles, and the rhomboid muscles, exert pressure lying in a weak compression effect range.

18. A garment according to claim 10, wherein the forearm portion includes a second piece of transverse compression effect covering the finger flexor muscles and exerting pressure lying in a strong compression effect range.

19. A garment according to claim 10, wherein the upper arm portion includes a second piece of transverse compression effect covering the biceps and triceps brachii muscles and exerting pressure lying in a weak compression effect range.

20. A garment according to claim 10, wherein the second piece of transverse compression effect on the back portion includes a mock seam so as to press said garment on the user's back, preferably along the spinal column.

21. A garment according claim 10, further comprising third pieces for covering the armpits and in the form of a net to encourage evacuating heat and perspiration.

22. A garment according to claim 1 in the form of tights comprising an anterior thigh portion, a posterior thigh portion, a knee portion, and a leg portion.

23. A garment according to claim 22, wherein the anterior thigh portion includes a second piece of transverse compression effect covering the rectus muscle, the lateral vastus muscle, and the adductor muscles or the medial vastus muscle.

24. A garment according to claim 22, wherein the posterior portion of the thigh includes a second piece of transverse compression effect covering the femoral biceps muscles and the adductor muscles or the semi-membraneous muscles.

25. A garment according to claim 22, wherein the knee portion includes a second piece of transverse compression effect covering with its anterior face the head of the fibula and with its posterior face the inside of the knee joint so as to extend over the top portion of the gastrocnemius, passing under said knee joint.

26. A garment according to claim 22, wherein said second pieces of longitudinal or transverse compression effect exert pressure lying in a weak pressure effect range.

27. A garment according to claim 22, wherein the leg portion includes a second piece of transverse compression effect covering the gastrocnemius muscles and the toe extensor muscles.

28. A garment according to claim 27, wherein said second piece of transverse compression effect exerts pressure lying in a strong compression effect range.

29. A garment according to claim 1 in the form of a long-sleeved costume comprising a forearm portion, an upper arm portion, a chest portion, a shoulder portion, a back portion, an abdomen portion, a buttocks portion, a thigh anterior portion, a thigh posterior portion, a knee portion, and a leg portion.

30. A garment according to claim 29, wherein the back portion includes a second piece of longitudinal compression effect covering the erector muscles of the spine and extending towards the bottom of the back over the low portion of the luteal muscle and the tensor muscle of the fascia lata, and towards the top of the back along the spinal column to be shared on either side of the nape of the neck and cover in particular the trapezius and rhomboid muscles, and in that said second piece exerts pressure lying in a weak compression effect range.

31. A garment according to claim 29 wherein the abdomen portion includes a second piece of longitudinal compression effect covering, on passing below the navel on either side of the waist, the internal oblique muscle and the rectus muscle, and the second piece exerts pressure lying in a weak compression effect range.

32. A garment according to claim 4, further comprising a first piece of basic elasticity covering the elbow joint, and in that said first piece is preshaped with an angle of 45° at the elbow joint, corresponding to the natural bending angle of the elbow.

33. (canceled)

34. (canceled)

35. A garment according to claim 10, further comprising basic elasticity covering the elbow joint, and in that said first piece is preshaped with an angle of 45° at the elbow joint, corresponding to the natural bending angle of the elbow.

36. A garment according to claim 29, further comprising a first piece of basic elasticity covering the elbow joint, and in that said first piece is preshaped with an angle of 45° at the elbow joint, corresponding to the natural bending angle of the elbow.
